(19) United States
(12) Patent Application Publication
(10) Pub. No.: US 2023/0148912 A1
(43) Pub. Date: May 18, 2023

(54) Deep Neural Network Systems, Devices, Methods and Media for Animal Health

(71) Applicant: Pet Metrics Inc., Dover, DE (US)

(72) Inventor: Gary A. Richter, Oakland, CA (US)

(21) Appl. No.: 17/977,897

(22) Filed: Oct. 31, 2022

Related U.S. Application Data
(60) Provisional application No. 63/278,980, filed on Nov. 12, 2021.
(60) Provisional application No. 63/391,189, filed on Jul. 21, 2022.

(51) Int. Cl. A61B 5/145; G16H 40/60; A61B 5/00

(52) U.S. Cl. CPC A61B 5/145; G16H 40/60; A61B 5/0022

(57) ABSTRACT

Exemplary embodiments include a system including an animal health device configured to receive animal health data, the animal health device including an array of non-contact transducers, lasers or sensors to detect the animal health data. The animal health data may include heart rate, heart rhythm, respiration rate, calories burned, an activity level, a sleep score, body temperature, or pulse oximetry and the animal health data may be received without touching a skin surface of an animal.

### CROSS-REFERENCE TO RELATED APPLICATIONS

The present application claims the priority benefit of U.S. Provisional Patent Application Ser. No. 63/278,980 filed on Nov. 12, 2021 titled “Systems, Methods and Media for Animal Health,” and the present application claims the priority benefit of U.S. Provisional Patent Application Ser. No. 63/391,189 filed on Jul. 21, 2022 titled “Deep Neural Network Systems, Methods and Media for Animal Health,” all of which are incorporated by reference in their entireties.

### FIELD OF INVENTION

The present technology pertains to deep neural network systems, devices, methods and media for animal health.

### SUMMARY OF EXEMPLARY EMBODIMENTS

Exemplary embodiments include a system having an animal health device configured to receive animal health data, the animal health device including an array of non-contact transducers, lasers or sensors to detect the animal health data. The animal health data may include heart rate, heart rhythm, respiration rate, calories burned, an activity level, a sleep score, body temperature, or pulse oximetry and the animal health data may be received without touching a skin surface of an animal.

The animal health device may be integrated onto a collar, harness or bridle or the animal health device may be attached to an existing collar, harness or bridle. The animal health device may include an accelerometer, a processor and/or a memory. The animal health device may be configured to connect to a network and/or configured to connect to a cloud resource, which according to various exemplary embodiments, may connect to a veterinary computing system and/or to an animal owner's computing system.

In various exemplary embodiments, the animal health data may be received with touching a skin surface of an animal and/or with touching the fur of an animal. The animal health data may be a predictor of a medical issue in an animal, such as a subtle increase in heart rate and a decrease in activity level to indicate the medical issue is pain. The predictor may be an increase in respiration rate when at rest to indicate the medical issue is heart or lung disease. The predictor may be an accelerometer indicating how much scratching activity is occurring when the animal owner is not around, and/or the accelerometer may indicate the animal has suffered a seizure. Additionally, the accelerometer may indicate the animal has separation anxiety. The animal health device, according to some exemplary embodiments, may be implantable and in some cases, may be a microchip.

In further exemplary embodiments, the system may be an intelligent secure networked messaging system configured by at least one processor to execute instructions stored in memory, including a data retention system and an analytics system, the analytics system performing asynchronous processing with a computing device and the analytics system communicatively coupled to a deep neural network, the deep neural network configured to receive a first input at an input layer, process the first input by one or more hidden layers, generate a first output and transmit the first output to an output layer, which may generate a first outcome. The first outcome may be transmitted to the input layer as input, and the first input may be first animal health data. Additionally, the first output may be a predictor of a medical issue in an animal and/or the first outcome may be second animal health data.

### BRIEF DESCRIPTION OF THE DRAWINGS

Certain embodiments of the present technology are illustrated by the accompanying figures. It will be understood that the figures are not necessarily to scale. It will be understood that the technology is not necessarily limited to the particular embodiments illustrated herein.

FIG. 1 is an exemplary architecture for an animal health device.

FIG. 2 illustrates an exemplary animal health device.

FIG. 3 shows an exemplary deep neural network.

FIG. 4 shows an exemplary method for using a deep neural network for animal health.

### DETAILED DESCRIPTION

The detailed embodiments of the present technology are disclosed here. It should be understood, that the disclosed embodiments are merely exemplary, which may be embodied in multiple forms. Those details disclosed herein are not to be interpreted in any form as limiting, but as the basis for the claims.

One of the biggest challenges faced by veterinary medicine is the speed at which medical problems are diagnosed. Obviously, all veterinary patients are non-verbal but that is only half of the issue. Evolutionarily, an animal that looks sick or injured is a target for predators. Because of that, even domestic animals will do everything they can to hide their illness or injury from their owner. By the time there is obviously something wrong, most animals have been sick for a long time. The result is most things in veterinary medicine are diagnosed very late in the course of disease—frequently too late to begin meaningful and effective treatment.

Data such as heart rate, heart rate variability, respiration rate, activity levels, calories burned, sleep scores, etc. can function as early predictors of medical issues in animals. For example, a subtle increase in heart rate with a decrease in activity level may indicate the animal is in pain. Increases in respiration rate when at rest are highly suggestive of heart or lung disease. The addition of an accelerometer can predict how much a pet is scratching when the owner is not around and even if the animal has a seizure. Measuring activity when the pet is home alone can be used to determine levels of separation anxiety. Information about a pet can be presented to an owner on an application on a portable computing device and can be transmitted to the animal's veterinarian. Cloud based AI can analyze data from an individual animal and provide predictive feedback to the owner and veterinarian that will allow for earlier intervention as well the ability to monitor an animal's response to a given therapy. Is the animal in less pain? Are they resting better? Are they more active? Some or all of these questions can be answered by the exemplary embodiments described herein.

According to various exemplary embodiments, AI will do much more than use data from an individual animal to predict their health status. It can also use the aggregate of thousands, eventually hundreds of thousands, of animals to improve predictability as well as generate health data using parameters such as age, sex, breed, size, etc. This kind of data can not only improve the quality of the information pet owners and veterinarians receive for individual animals, but these datasets can be invaluable for researchers who are evaluating efficacy of new pharmaceuticals, supplements, and treatments. The more animals using the systems, methods and media described herein, the greater, and more valuable, the data becomes.

The exemplary systems, methods and media described herein can also be used in the multi-billion dollar performance horse industry, where fractions of a second are the difference between winning and losing. Additionally, this has applications in food animal production. Agriculture such as dairy production is a very thin margin business that relies on volume to be profitable. Increasing the amount of milk produced per day or the production lifespan of a dairy cow has profit potential that is difficult to even quantify.

There is no HIPPA in veterinary medicine. Imagine the value in approaching a company that makes a product for arthritic dogs and offering them a dataset that includes tens of thousands of people who own arthritic dogs so they can improve their products.

The trajectory of biometric technology is nearly vertical. A “new and improved” microchip that will keep pets healthy will have enormous demand.

After collecting data, the datasets will begin to grow and evolve. Exemplary systems, methods and media include a device that collects non-contact vital signs.

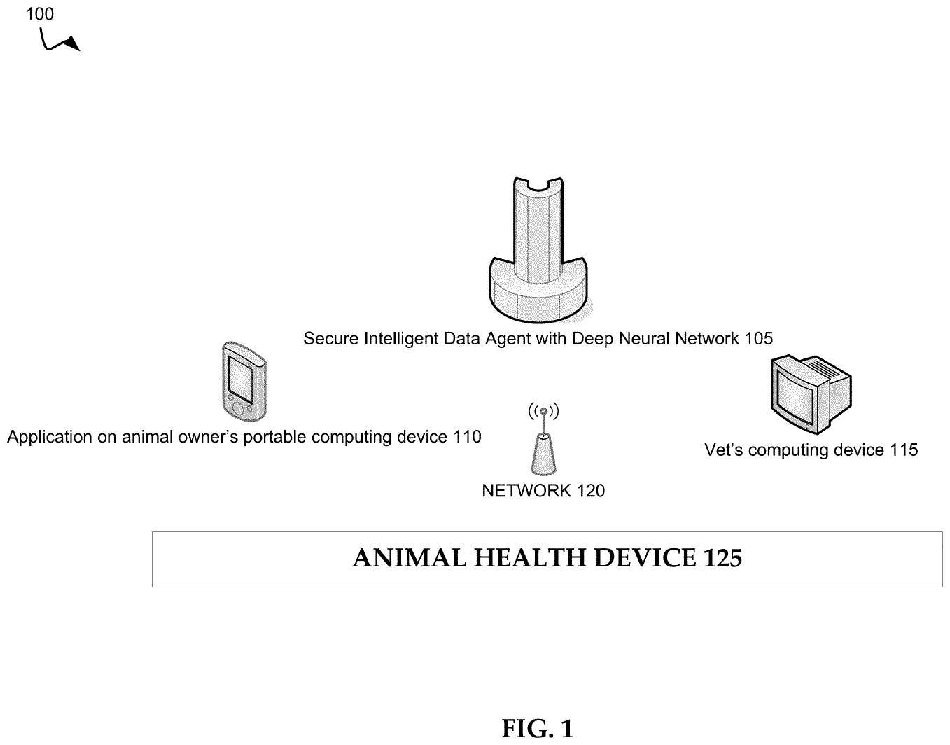

FIG. 1 is an exemplary architecture for an animal health device.

Shown in exemplary architecture 100 is secure intelligent data agent with a deep neural network 105, application on animal owner's portable computing device 110, veterinarian's computing device 115, network 120 and animal health device 125.

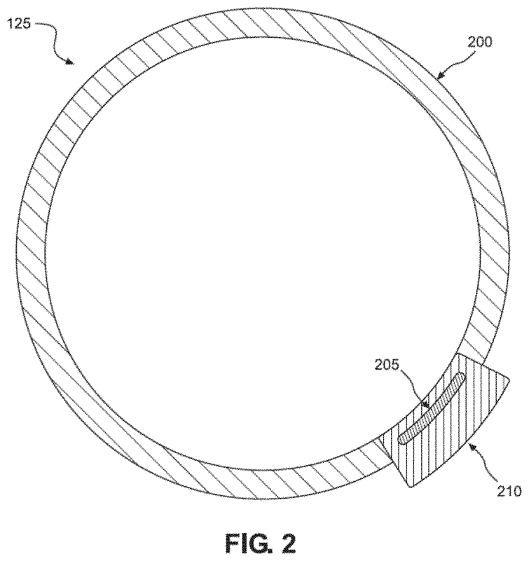

According to various exemplary embodiments, the secure intelligent data agent with a deep neural network 105 may comprise specialized dedicated processors. It may or may not reside in the cloud. The application on an animal owner's portable computing device 110 may be downloaded from an application store or a website. Additionally, a website may host a version of the application. The veterinarian's computing device 115 may be configured with computing resources targeted specifically for veterinarians. The network can be any number of networks, including Internet, cell-based, local area network, etc. The animal health device 125 is described in greater detail in connection with FIG. 2.

In some exemplary embodiments, the secure intelligent data agent with a deep neural network 105 may be configured with a deep neural network for processing data received from various sources, including the animal health device 125. A neural network is a framework of machine learning algorithms that work together to classify inputs based on a previous training process. Additionally, the animal health device 125 may be configured with a memory, processor and/or transmission means for sending stored data when the animal health device is outside of a particular network.

FIG. 2 illustrates an exemplary animal health device.

Shown as part of animal health device 125 (FIG. 1) in FIG. 2 is collar 200 (can also be a harness or bridle), array of non-contact transducers, lasers, and/or sensors 205 to detect heart rate, heart rhythm, body temperature, pulse oximetry without directly touching the skin, and device 210 to either be integrated into a collar, harness, or bridle, or attached to an existing collar, harness or bridle.

In an alternative embodiment, the same or similar technology may be applied to humans. For example, using various light waves, human vitals may be taken 7 9907US without the need to contact a person's skin. Such vitals may even be taken through clothing. The same analytical tools as described herein may also be applied, in compliance with the Health Insurance Portability and Accountability Act of 1996 (“HIPAA”).

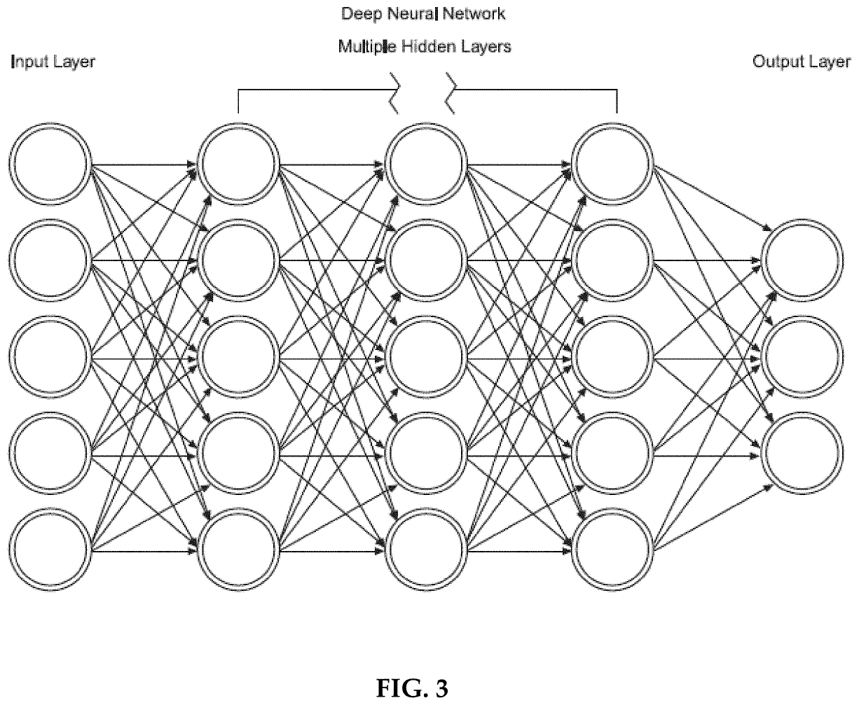

FIG. 3 shows an exemplary deep neural network.

Deep neural networks, also known as artificial neural networks (ANNs) or simulated neural networks (SNNs), are a subset of machine learning and are at the heart of deep learning algorithms. Their name and structure are inspired by the human brain, mimicking the way that biological neurons signal to one another. Artificial neural networks (ANNs) are comprised of node layers, comprising an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed along to the next layer of the network.

Neural networks rely on training data to learn and improve their accuracy over time. However, once these learning algorithms are fine-tuned for accuracy, they are powerful tools in computer science and artificial intelligence, allowing one to classify and cluster data at a high velocity. Tasks in speech recognition or image recognition can take minutes versus hours when compared to the manual identification by human experts. One of the most well-known neural networks is Google™'s search algorithm.

In some exemplary embodiments, one should view each individual node as its own linear regression model, composed of input data, weights, a bias (or threshold), and an output. Once an input layer is determined, weights are assigned. These weights help determine the importance of any given variable, with larger ones contributing more significantly to the output compared to other inputs. All inputs are then multiplied by their respective weights and then summed. Afterward, the output is passed through an activation function, which determines the output. If that output exceeds a given threshold, it “fires” (or activates) the node, passing data to the next layer in the network. This results in the output of one node becoming the input of the next node. This process of passing data from one layer to the next layer defines this neural network as a feedforward network. Larger weights signify that particular variables are of greater importance to the decision or outcome.

Deep neural networks, according to various exemplary embodiments, are feedforward, meaning they flow in one direction only, from input to output. However, one can also train a model through backpropagation; that is, move in the opposite direction from output to input. Backpropagation allows one to calculate and attribute the error associated with each neuron, allowing one to adjust and fit the parameters of the model(s) appropriately.

In machine learning, backpropagation is an algorithm for training feedforward neural networks. Generalizations of backpropagation exist for other artificial neural networks (ANNs), and for functions generally. These classes of algorithms are all referred to generically as “backpropagation”. In fitting a neural network, backpropagation computes the gradient of the loss function with respect to the weights of the network for a single input-output example, and does so efficiently, unlike a naive direct computation of the gradient with respect to each weight individually. This efficiency makes it feasible to use gradient methods for training multilayer networks, updating weights to minimize loss; gradient descent, or variants such as stochastic gradient descent, are used. The backpropagation algorithm works by computing the gradient of the loss function with respect to each weight by the chain rule, computing the gradient one layer at a time, iterating backward from the last layer to avoid redundant calculations of intermediate terms in the chain rule; this is an example of dynamic programming. The term backpropagation strictly refers only to the algorithm for computing the gradient, not how the gradient is used; however, the term is often used loosely to refer to the entire learning algorithm, including how the gradient is used, such as by stochastic gradient descent. Backpropagation generalizes the gradient computation in the delta rule, which is the single-layer version of backpropagation, and is in turn generalized by automatic differentiation, where backpropagation is a special case of reverse accumulation (or “reverse mode”).

With respect to FIG. 3, according to exemplary embodiments, the system produces an output, which in turn produces an outcome, which in turn produces an input. In some embodiments, the output may become the input.

According to various exemplary embodiments, the input may include heart rate, heart rhythm, movement data, GPS data, sound data including barking, respiration rate and/or temperature. Calculated data based on this may include respiration rate (if not directly measured), heart rate variability, and a determination if a pet is scratching, has had a seizure and/or has separation anxiety.

The input in various exemplary embodiments may include owner input such as appetite, type of food and amount fed, vomiting, diarrhea, coughing, sneezing, limping, seizure, time exercised, type of exercise, overall demeanor, body weight, body condition score (measure of over/underweight), medication schedule, and/or vaccine or other medical reminders.

Other parameters monitored and output may include, according to exemplary embodiments, calories burned, appropriate amount to feed based on calories burned, recommended amount of exercise, sleep/restfulness scores, activity scores, guidance regarding issues detected either from device and/or owner input, including vomiting, diarrhea, coughing, sneezing, limping, seizure, separation anxiety, changes in biometric parameters based in changes such as medication, supplement, diet change, etc. Various exemplary embodiments may objectively quantify what is working and what is not working and/or provide reminders when things are due such as medications, grooming, time for a walk, etc.

Various exemplary embodiments may provide healthy monitoring of biometric parameters to establish profiles for an individual and for a database of animals based on species, breed, sex, spayed/neutered, age, and/or activity level. Various exemplary embodiments may provide known diagnosis requiring monitoring of biometric parameters to provide insight into treatment monitoring and providing information to contribute to treatment plans. Performance monitoring may include monitoring competitive athletic animals and/or working animals to achieve better performance and prevent injury.

Here, the deep neural network may have a wealth of collected information, including the above information. In turn, the deep neural network may generate output information to formulate a strategy. Application of the strategy may result in an outcome that may be fed back into the deep neural network for formulating an improved strategy, which may lead to improved outcomes.

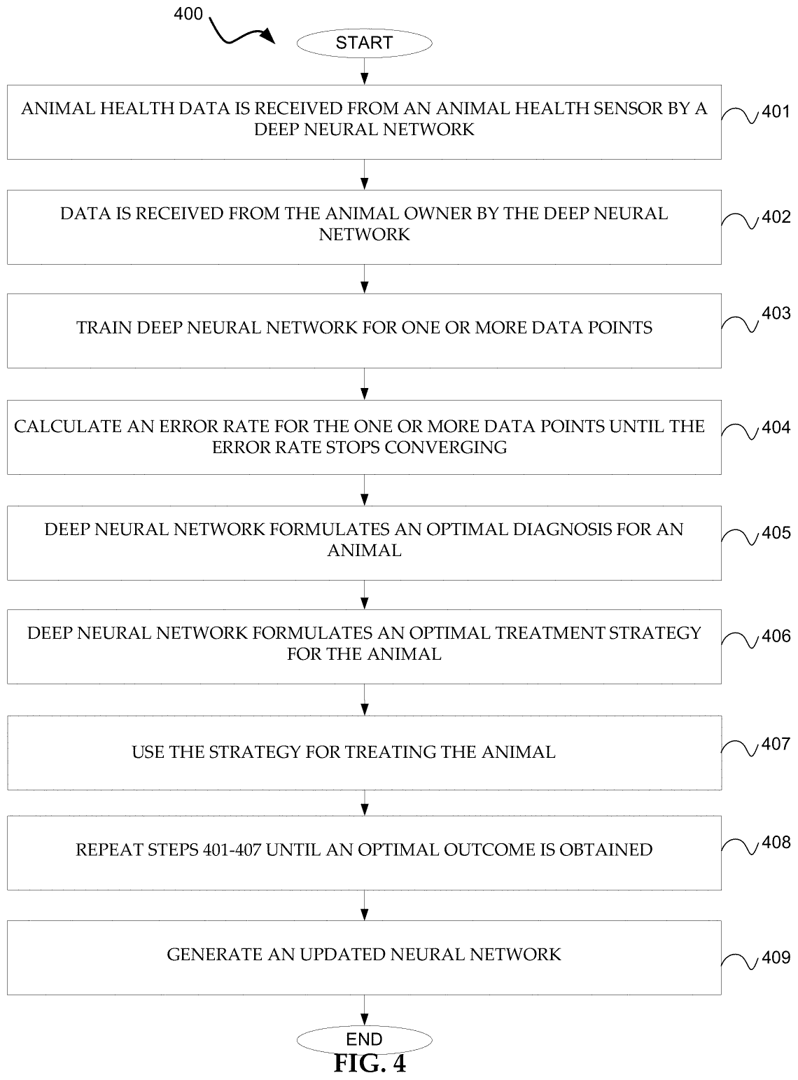

FIG. 4 shows an exemplary method for using a deep neural network for animal health. Method 400 comprises steps 401 through 409.

At step 401, animal health data is received from an animal health sensor by a deep neural network.

At step 402, data is received from the animal owner by the deep neural network.

At step 403, the deep neural network is trained for one or more data points. For example, if the animal health data indicates an animal is exhibiting separation anxiety and the data received from the animal owner indicates the animal is at a pound, the deep neural network will likely correctly conclude that the animal is indeed at a pound and not enjoying it.

At step 404, an error rate is calculated for one or more data points until the error rate stops converging. For example, at step 403 the situation was correctly determined for the two data points and therefore the error rate stopped converging.

At step 405, the deep neural network formulates an optimal diagnosis for the animal. For example, the animal has separation anxiety.

At step 406, the deep neural network formulates an optimal treatment strategy for the animal. For example, the animal should be walked and petted before it can return home to its owner.

At step 407, the strategy is used for treating the animal. For example, the animal is walked and petted before returning home to its owner.

At step 408, steps 401-407 are repeated until an optimal outcome is obtained. For example, the animal is no longer showing separation anxiety.

At step 409, the deep neural network is updated. As a result, it will spot separation anxiety much more quickly and also learn how to further differentiate other diagnosis from one another and how to develop better treatment strategies much faster on the deep neural network and further improve animal health outcomes.

While various embodiments have been described above, it should be understood that they have been presented by way of example only, and not limitation. The descriptions are not intended to limit the scope of the present technology to the particular forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A system comprising:
an animal health device configured to receive animal health data, the animal health device including an array of non-contact transducers, lasers or sensors to detect the animal health data.

2. The system of claim 1, further comprising the animal health data including heart rate, heart rhythm, respiration rate, calories burned, an activity level, a sleep score, body temperature, or pulse oximetry.

3. The system of claim 2, wherein the animal health data is received without touching a skin surface of an animal.

4. The system of claim 1, wherein the animal health device is integrated onto a collar, harness or bridle.

5. The system of claim 1, wherein the animal health device is attached to an existing collar, harness or bridle.

6. The system of claim 1, further comprising an accelerometer.

7. The system of claim 1, further comprising the animal health device having a processor and a memory.

8. The system of claim 1, further comprising the animal health device configured to connect to a network.

9. The system of claim 8, further comprising the animal health device configured to connect to a cloud resource.

10. The system of claim 9, further comprising the cloud resource configured to connect to a veterinary computing system or an animal owner's computing system.

11. The system of claim 2, wherein the animal health data is received with touching a skin surface of an animal.

12. The system of claim 2, wherein the animal health data is received with touching fur of an animal.

13. The system of claim 1, further comprising the animal health data is a predictor of a medical issue in an animal.

14. The system of claim 13, wherein the predictor is a subtle increase in heart rate and a decrease in activity level to indicate the medical issue is pain.

15. The system of claim 13, wherein the predictor is an increase in respiration rate when at rest to indicate the medical issue is heart or lung disease.

16. The system of claim 13, wherein the predictor is an accelerometer indicating how much scratching activity is occurring when the animal owner is not around.

17. The system of claim 13, wherein the predictor is an accelerometer indicating the animal has suffered a seizure.

18. The system of claim 13, wherein the predictor is an accelerometer indicating the animal has separation anxiety.

19. The system of claim 1, wherein the animal health device is implantable.

20. The system of claim 19, wherein the implantable health device is a microchip.

21. An intelligent secure networked messaging system configured by at least one processor to execute instructions stored in memory, the system comprising:
a data retention system and an analytics system, the analytics system performing asynchronous processing with a computing device and the analytics system communicatively coupled to a deep neural network;
the deep neural network configured to:
receive a first input at an input layer;
process the first input by one or more hidden layers;
generate a first output; and
transmit the first output to an output layer.

22. The intelligent secure networked messaging system of claim 21, further comprising generating a first outcome.

23. The intelligent secure networked messaging system of claim 22, further comprising the first outcome being transmitted to the input layer as input.

24. The intelligent secure networked messaging system of claim 21, further comprising the first input being first animal health data.

24. The intelligent secure networked messaging system of claim 21, further comprising the first output being a predictor of a medical issue in an animal.

25. The intelligent secure networked messaging system of claim 22, further comprising the first outcome being second animal health data.
